(19) United States
(12) Patent Application Publication
(10) Pub. No.: US 2009/0181070 A1
(43) Pub. Date: Jul. 16, 2009

(54) Emulsification Systems, Umulsions and Wet Wipes Containing Such Emulsions

(75) Inventors: Trevor Graham Blease, Cleveland (GB); Francisus Johannes Groenhof, Bunnik (NL); Graham Timothy Pennick, North Yorkshire (GB)

Correspondence Address: JONES DAY, 222 EAST 41ST ST, NEW YORK, NY, 10017, US

(73) Assignees: Croda International PLC, Goole, North Humberside (GB); Uniqeme B.V., Gouda (NL)

(21) Appl. No.: 12/223,692

(22) PCT Filed: Jan. 19, 2007
(86) PCT No.: PCT/GB2007/000143

§ 371 Date: Dec. 4, 2008

(30) Foreign Application Priority Data
Feb. 8, 2006 (GB) 0602528.2

(51) Int. Cl. A61K 8/89; B01F 17/54; A61K 8/06

(52) U.S. Cl. 424/443; 516/55; 424/78.02

(57) ABSTRACT

A method of making an oil-in-water emulsion is described in which an oil emulsifier and a polysaccharide emulsion stabiliser, which stabiliser comprises a Xanthan polysaccharide and a polyglucomannan polysaccharide, is dispersed in an oil. The oil-based dispersion so formed is then combined with water, preferably at low temperature and under low shear conditions, to form an oil-in-water emulsion. The polysaccharide emulsion stabiliser and optionally oil emulsifier may be dispersed directly into a relatively polar oil to form the oil-based dispersion or, alternatively, into a relatively polar non-aqueous liquid medium, which medium then being mixed with the oil before it is combined with the water to form the emulsion. The oil-in-water emulsions are particularly useful in personal care and cosmetic applications such as sprays, body moisturisers, sun screens and wet wipes for cosmetic or personal hygiene cleaning uses.

The invention relates to emulsification systems, emulsions, wet wipes containing such emulsions and to methods of making emulsions.

Oil-in-water emulsions are widely used in the personal care and cosmetic industry to deliver ingredients to skin and hair, either by direct application thereto of milks or lotions or through the medium of wet wipes and other similar applications.

Personal care emulsion products such as creams and milks desirably have a number of properties in combination: stability in manufacture, formulation, storage and use; a viscosity appropriate to the end use; and preferably a desirable body and good skin feel. Body and skin feel are usually assessed subjectively, and although good body and/or skin feel are commonly associated with a non-Newtonian, shear thinning viscosity profile, a shear thinning profile does not guarantee a good body or skin feel. Typical conventional personal care emulsion products use emulsifiers (including emulsion stabilisers) in amounts of about 3 to about 5% by weight of the emulsion. Recently, thickeners have been proposed as emulsion stabilisers and the mechanism of stabilisation when these are used appears to be that the thickener increases the low shear viscosity of the emulsion sufficiently to provide a barrier to emulsion droplet coalescence, probably by limiting the movement of the droplets.

A particularly effective emulsification system is described in EP 1137396 B1, which is incorporated herein in its entirety by reference, wherein certain combinations of high molecular weight polysaccharides provide good emulsion stabilisation at levels that do not give high, or even significantly increased, low shear viscosity and that, by using such combinations, the amount of emulsifier, usually a relatively low molecular weight, often non-ionic, surfactant, can be much less than is used conventionally in emulsions, particularly emulsions for personal care products such as cosmetic skin creams and milks.

Thus, EP 1137396 B1 discloses personal care or cosmetic oil-in-water emulsions in which an emulsifier stabiliser system comprises an emulsifier for the oil and a polysaccharide combination of a Xanthan polysaccharide and a polyglucomannan polysaccharide. The combined amount of emulsifier and polysaccharide stabiliser in such emulsions can be much lower than the typical 3 to 5% used in conventional personal care emulsion systems. In particular, in many emulsions disclosed in EP 1137396 B1, the amount of emulsifier can be less than about 1.5%, particularly up to about 1%, and the amount of polysaccharide stabiliser can be less than about 0.5%, and sometimes as little as about 0.02%, desirably with the combined amount being less than about 1.5%, particularly up to about 1%. The minimum amount of emulsifier is typically about 0.02% more usually 0.025% by weight of the emulsion.

The preparation of emulsions as disclosed in EP 1137396 B1 requires the emulsion/polysaccharide stabiliser mixture to be heated above about 60° C. in the aqueous phase and/or subjected to high intensity mixing to ensure the polysaccharide stabiliser functions effectively. Although phase inversion emulsification, in which the emulsifier and polysaccharide stabiliser is initially mixed into the oil phase, is mentioned as an option in EP 1137396 B1, it is still necessary to have the heating and/or high intensity mixing step in the emulsification process. The normal practise is to mix the emulsifier and polysaccharide stabiliser into the aqueous phase, for example as disclosed in US 2005/0009431 A1 wherein, in the examples, Arlatone V 175™ emulsifier/polysaccharide stabiliser material within the scope of EP 1137396 B1 is mixed into the aqueous phase using a triblender.

In a similar disclosure to EP 1137396 B1, WO 01/96461 A1 discloses initially making a fluid gel using Xanthan and one or more non-gelling polysaccharides such as galactomannans and gluccomannans by heating an aqueous mixture of Xanthan and the non-gelling polysaccharide to temperatures greater than 50° C. and allowing it to cool whilst subjecting it to shear. The fluid gels are then used in personal care and cosmetic formulations including those using oil-in-water emulsions.

One use of oil-in-water emulsions is in the preparation of wet wipes for skin cleansing. The aforementioned 2005/0009431 A1, together with US 2005/0008680 A1 and US 2005/0008681 A1 published on the same day, which are incorporated herein in their entirety by reference, provide a useful background to the uses to which wet wipes are put as well as the materials that they are made of.

US 2005/0009431 A1 describes a method of making a wet wipe in which a concentrated emulsion composition consisting of emollient, surfactant and not more than about 30% by weight of water, is made initially and the concentrate is then diluted down with further water to become an oil-in-water emulsion before being applied to a wipe substrate.

As will be appreciated, wet wipes are a high volume consumer product used for a wide variety of cleansing purposes. Consequently, the high energy requirements for the manufacture of suitable oil-in-water emulsions for use in making the wipes requires a high capital investment as well as high manufacturing costs—as is recognised in US 2005/0009431 A1. Even the process described in US 2005/0009431 A1, whilst avoiding some of the costs inherent in the earlier manufacturing methods, still involves a two-stage process, additional emulsifier costs (in making the initial concentrate) and additional equipment and running costs (for the inclusion of products of the type described in EP 1137396 B1).

The Applicant has now found that the aforementioned disadvantages can be significantly reduced or overcome using the present invention in which surprisingly oil-in-water emulsions using an oil emulsifier and a polysaccharide emulsion stabiliser can be made without the need to heat the emulsion or subject it to high shear.

More particularly, according to the present invention, a method of making an oil-in-water emulsion comprises dispersing an oil emulsifier and a polysaccharide emulsion stabiliser, which stabiliser comprises a Xanthan polysaccharide and a polyglucomannan polysaccharide, in an oil, combining the oil-based dispersion so formed with water, preferably at low temperature and under low shear conditions, to form an oil-in-water emulsion.

The Applicant has found that a polysaccharide emulsion stabiliser and optionally oil emulsifier may be effectively dispersed in oils that are relatively polar. Alternatively, when the oil(s) are relatively non-polar, the polysaccharide emulsion stabiliser and optionally oil emulsifier may be effectively dispersed in a relatively polar non-aqueous liquid medium, which medium being mixed with the oil prior to the step of combining the oil-based dispersion with the water to form the emulsion. If required, the dispersion of the polysaccharide emulsion stabiliser and optionally oil emulsifier in a relatively polar non-aqueous liquid medium may also be used even when the oil is relatively polar. The oil emulsifier may be dispersed separately in the non-polar oil, polar oil and/or non-aqueous liquid medium as required, but is preferably dispersed together with and in the same oil/medium as the polysaccharide emulsion stabiliser.

A measure of the polarity of the oil or the non-aqueous medium may be obtained using solubility parameters. A widely used solubility parameter is the Hansen and Beerbower solubility parameter as described in A F M Barton, CRC Handbook of Solubility parameters and other cohesion parameters, CRC press, 1983 p. 85-87 and A F M Barton in Chemical Reviews, 1975, Vol 75 No. 6 p. 731-753.

Accordingly, in a more specific embodiment of the present invention, a method of making an oil-in-water emulsion comprises the steps of:
(a) dispersing a polysaccharide emulsion stabiliser and optionally an oil emulsifier, wherein said polysaccharide emulsion stabiliser comprises a Xanthan polysaccharide and a polyglucomannan polysaccharide, either in:
   (i) an emollient oil with a Hansen and Beerbower solubility parameter of at as 19 but less than 45; or
   (ii) a non-aqueous liquid medium with a Hansen and Beerbower solubility parameter of at least 19 but less than 45 and combining the dispersion in said medium with an oil that has a Hansen and Beerbower solubility parameter of at least 19 but less than 45; or
   (iii) a non-aqueous liquid medium with a Hansen and Beerbower solubility parameter of at least 19 but less than 45 and combining the dispersion in said medium with an oil that has a Hansen and Beerbower solubility parameter of less than 19;
(b) combining water with the oil-based dispersion formed in step (a), preferably at low temperature and under low shear conditions, to form an oil-in-water emulsion, and
(c) continuing to mix the emulsion formed in step (b), preferably under low shear conditions, until homogenous.

It will be understood that, for certain classes of emollient oils, for example silicone oils, it is not possible to assign a Hansen and Beerbower solubility parameter. In such instances, in accordance with the method of the present invention, the emollient oil is treated as though it has a Hansen and Beerbower solubility parameter of less than 19 and a dispersion is made in accordance with step (a)(iii) in the preceding paragraph. For the avoidance of doubt, emollient oils that do not have Hansen and Beerbower solubility parameter assigned or assignable to them are treated for the purposes of this specification, including the claims, as having a Hansen and Beerbower solubility parameter of less than 19 and, thus, are intended to be within the relevant wording used in the specification, including the claims.

Also, according to the present invention, a method of making a wet wipe comprises the steps of:
1. making an oil-in-water emulsion in accordance with the invention as hereinbefore described; and
2. providing a wipe substrate and applying a quantity of the oil-in-water emulsion thereto.

By the term “low temperature” as used in the methods of the present invention is meant a temperature of not more than 50° C., preferably not more than 40° C. and especially not more than 30° C. Additionally, the term “low temperature” as used in the methods of the present invention means a temperature of greater than 0° C., preferably at least 10° C., and especially at least 15° C. Preferred temperatures for use in the methods of the present invention are in the range 15° C. to 30° C., more especially in the range 20° C. to 25° C.

By the term “low shear” as used in the methods of the present invention is meant a shear rate of not more than 5000 s⁻¹. Additionally, the term “low shear” as used in the methods of the present invention means a shear rate of greater than 10 s⁻¹, preferably at least 50 s⁻¹.

No special mixing device is required to combine the solid additives and emollient oil as the emulsifier and polysaccharide emulsion stabiliser are readily dispersed (either in the emollient oil or in the non-aqueous medium and then subsequently the emollient oil) even when using conventional “low shear” mixing devices such as vessels with internal rotors (eg anchor, flat blade, angled blade, propeller agitators with two or more blades) operating at rotational speeds of less than 1000 rpm, preferably less than 500 rpm, with a specific power input of less than 1000 W/m3 of total vessel volume, or vessels operating with jet mixers or external piping mixing devices operating on a pump-round loop or in a continuous flow configuration. The same types of simple mixing system are sufficient to carry out all the process steps from initial dispersion preparation to final emulsion.

The initial volumes of emulsifier/stabiliser, non-aqueous liquid medium (when present) and emollient oil are relatively low compared to the final emulsion volume. Therefore, it is necessary to ensure that the mixing device is in contact with the liquids for the initial production steps. This can be achieved by the use of several agitator blades situated at different heights up the vessel, such that the lowest blade provides the necessary agitation for the initial dispersion; by mounting a single impeller sufficiently close to the vessel base such that it is in contact with the initial volume; by using several vessels of increasing size in sequence for the production steps; by employing one or more pump-round loops of different volumes external to the vessel with jet mixing or other in-line mixing devices (eg static mixers, or powered rotating mixing devices); by using similar continuous in-line mixing techniques in a piping system or any combination of these together or in isolation. As will be appreciated, such systems may be operated either on a batch basis or a continuous basis depending on the configuration of the equipment used.

Suitable non-aqueous liquid media for use in the present invention as described above may be selected from relatively polar emollient oils (preferably with a Hansen and Beerbower solubility parameter of at least 19 but less than 45), alcohols, glycols and glycerine and mixtures thereof. Examples of suitable emollient oils are stearyl alcohol 15-propoxylates (solubility parameter 20.8), propylene glycol isostearate (solubility parameter 19.54) and a mixture of triethylhexanoin and isopropyl isostearate (solubility parameter 19.04). Examples of other non-aqueous liquid media are glycerine (solubility parameter 38) and propylene glycol (solubility parameter 31). It will be understood that any such non-aqueous media have to be suitable for use in personal care applications. Preferably, the non-aqueous media are those registered as solvents by the Cosmetics Toiletries and Fragrance Association. A particularly preferred non-aqueous liquid medium is propylene glycol.

Whilst small amounts of water, ie not greater than 1% by weight, may be present in the oil phase made in step (b) or step 1(b) above, preferably the oil phase is essentially water free; although it will be appreciated any residual water (moisture) content in the components thereof will be present.

Alternatively, if desired, concentrates, ie water-in-oil emulsions, may be made. Such concentrates may contain at least 70% by weight of oil, more preferably at least 80% by weight of oil. Such concentrated emulsions may be used as pre-manufactured concentrates for combination with water, and other components if desired, using the methods of the invention to make product emulsions.

Preferably, the amount of emulsifier added to the emollient oil or non-aqueous medium in the methods of the invention is such that the amount of emulsifier in the final emulsion is not more than about 1.5%, particularly not more than about 1%, by weight of the final emulsion. Similarly, the amount of polysaccharide stabiliser added to the emollient oil or non-aqueous medium in the methods of the invention is such that the amount of polysaccharide stabiliser in the final emulsion is not more about 0.5%, and sometimes as little as about 0.01% by weight of the final emulsion. Preferably, the combined amount of emulsifier and polysaccharide stabiliser added to the emollient oil or non-aqueous medium in the methods of the invention is such that the amount of emulsifier and polysaccharide stabiliser in the final emulsion is not more than about 1.5%, particularly not more than about 1%, by weight thereof. The minimum amount of emulsifier in the final emulsion is typically about 0.05% by weight of the emulsion.

More particularly, the amount of emulsifier/polysaccharide stabiliser combination added to the non-aqueous medium in the methods of the invention is in the range 1% to 20% by weight based on the weight of the non-aqueous medium and emollient oil, more preferably, in the range 5% to 15% by weight based on the weight emollient oil or non-aqueous medium.

The amount of non-aqueous medium added to the emollient oil in the methods of the invention is such that the amount of non-aqueous medium in the final emulsion is preferably not more than about 5%, more preferably not more than about 3%, and especially not more than about 2%, by weight of the final emulsion. Preferably, the amount of non-equeous medium added to the emollient oil in the methods of the invention is such that the amount of non-aqueous medium in the final emulsion is preferably not less than about 0.25%, more preferably not less than about 0.5%, by weight of the final emulsion.

More particularly, the amount of non-aqueous medium added to the emollient oil in step (b) or step 1(b) above is in the range 10% to 80% by weight based on the weight of the emollient oil, more preferably, in the range 20% to 50% by weight based on the weight of the emollient oil.

The invention includes an emollient oil composition comprising emollient oil and non-aqueous medium in which is dispersed oil emulsifier and a polysaccharide emulsion stabiliser wherein said polysaccharide emulsion stabiliser comprises a Xanthan polysaccharide and a polyglucomannan polysaccharide.

The amount of oil to which water is added in the methods of the present invention to form the final emulsion will depend upon the use to which the emulsion will be put. For example, personal care emulsions can be divided by viscosity into milks and lotions, which typically have a low shear viscosity of up to about 10000 mPa.s, and creams which typically have a low shear viscosity of more than about 20000 mPa.s. Typically, milks and lotions have a low shear viscosity of from about 100 to about 10000 mPa.s, more usually from about 500 to about 5000 mPa.s, and typically creams have a low shear viscosity of at least about 30000 mPa.s, particularly from about 30000 to about 80000 mPa.s, although even higher viscosities e. g. up to about 10⁶ mPa.s, may also be used. In this context low shear viscosity refers to viscosity measured at shear rates of about 0.1 to 10 s⁻¹ as is typically used in Brookfield viscometers. Because for good skin feel, personal care and cosmetic emulsions are usually shear thinning, the measured low shear viscosity is only a general guide to whether the product is a milk (or lotion) or cream.

Products made by the methods of the present invention includes both milk (and lotion) and cream emulsions and specifically include personal care or cosmetic oil-in-water emulsion milks or lotions having a low shear viscosities of up to about 10000 mPa.s, which includes as an emulsifier stabiliser system an emulsifier for the oil and a polysaccharide stabiliser. Such products further includes personal care or cosmetic oil-in-water cream emulsions having a low shear viscosity of more than about 20000 mPa.s, which includes as an emulsifier stabiliser system an emulsifier for the oil and a polysaccharide stabiliser, the emulsion further including thickener components.

More particularly, when the emulsions made by the methods of the present invention are intended for application to wipe substrates, the oil-in-water emulsions, which include an emulsifier for the oil and a polysaccharide stabiliser, have low shear viscosities of not more than about 5000 mPa.s, more especially not more than about 4000 mPa.s. Such oil-in-water emulsions have low shear viscosities of not less than about 100 mPa.s, more especially not less than about 200 mPa.s

The use of even very low concentrations of polysaccharide stabiliser e. g. as low as about 0.01% by weight of the emulsion, can give useful improvements in emulsion stability. In practice, the amount of the polysaccharide stabiliser used will be chosen to give emulsions with extended stability and will generally be at least about 0.02% by weight of the emulsion. The maximum concentration generally used depends on the emulsion system, but typically a concentration of about 0.1% by weight of the emulsion is employed. Thus, suitable general concentration ranges are from about 0.02% to about 0.5%, more usually from 0.025 to about 0.25%, particularly up to about 0.2% and especially from 0.025 to 0.15%, by weight of the emulsion. Relatively high concentrations in these ranges may be used, eg where particularly difficult emulsions are being made including those using very hydrophobic oils, or and especially in cream formulations, or where electrolyte may be present (see below), even though the rheology may not be ideal.

The emulsions made and stabilised according to the invention can have exceptionally high stability even at elevated temperatures eg up to about 50° C. However, the polysaccharide combinations are sensitive to ionic materials which act to destabilise the emulsions. For this reason, ionic materials e. g. acids, bases and salts including neutral salts, such as organic or inorganic salts, are desirably present only at low concentrations in the emulsions of this invention, or are absent. Generally the concentration of ionic materials will be not greater than about 0.05 molar, desirably not more than about 0.02 molar and particularly not more than about 0.01 molar. Similarly, ionic surfactants, including emulsifiers, including anionic, cationic and zwiterionic surfactants are desirably not present at significant concentrations in the emulsions of the invention. Amphoteric surfactants can be used, but usually only under conditions where they do not support charged species and, as this tends to be an environment where amphoteric surfactants are not particularly effective, they are not usually desirably included.

Accordingly, the oil emulsifier used in the invention is desirably one or more non-ionic emulsifier(s). Suitable emulsifiers include conventional non-ionic oil-in-water emulsifier surfactants such as alkoxylate emulsifiers and surfactants that can be derived from natural materials such as fatty acid esters, ethers, hemi-acetals or acetals of polyhydroxylic compounds. The specific nature of the emulsifier surfactant used in any particular instance depends on the type of emulsion being made, particularly whether fatty amphiphilic thickeners are being used, the degree of stability required, the nature of the oil being emulsified and the total desired level of emulsifier/stabiliser system.

The term alkoxylate emulsifier is used to refer to surfactants in which a hydrophobe, usually a hydrocarbyl group, is connected through the residue of a linking group having a reactive hydrogen atom to an oligomeric or polymeric chain of alkylene oxide residues. The hydrocarbyl group is typically a chain, commonly an alkyl chain, containing from 8 to 24, particularly 12 to 22, and usually 14 to 20 carbon atoms. The linking group can be an oxygen atom (hydroxyl group residue); a carboxyl group (fatty acid or ester residue); an amino group (amine group residue); or a carboxyamido (carboxylic amide residue). The alkylene oxide residues are typically residues of ethylene oxide (C₂H₄O) or propylene oxide (C₃H₈O) or combinations of ethylene and propylene oxide residues. When combinations are used the proportion of ethylene oxide residues will usually be at least about 50 mole % and more usually at least 75 mole %, the remainder being propylene oxide residues. Particularly and desirably, substantially all the residues are ethylene oxide residues. The number of alkylene residues in the emulsifier molecule is desirably from 2 to about 200.

In this aspect the invention includes making low viscosity milk emulsions and higher viscosity cream emulsions. Specifically, the invention includes making a personal care or cosmetic oil in water emulsion milk having a viscosity of up to about 10000 mPa.s, which includes as an emulsifier stabiliser system an emulsifier for the oil, which is a fatty acid ester, ether, hemi-acetal or acetal of a polyhydroxylic compound, in an amount of from about 0.5 to about 1.5% by weight of the emulsion and a polysaccharide stabiliser in an amount of from about 0.02 to about 0.25% by weight of the emulsion. The invention further specifically includes making a personal care or cosmetic oil in water cream emulsion having a viscosity of more than about 20000 mPa.s, which includes as an emulsifier stabiliser system an emulsifier for the oil which is a fatty acid ester, ether, hemi-acetal or acetal of a polyhydroxylic compound, in an amount of from about 0.5 to about 1.5% by weight of the emulsion and a polysaccharide stabiliser in an amount of from about 0.02 to about 0.25% by weight of the emulsion, the emulsion further including thickener components.

It can be useful to use a combination of different types of emulsifier and in particular to combine hydrophilic emulsifiers ie having a high Hydrophile Lipophile Balance (HLB), eg more than about 12, and hydrophobic emulsifiers, ie having a low HLB, eg less than about 8, in making the emulsions of the invention. Relatively hydrophilic emulsifiers include alkoxylate emulsifiers with an average of from about 10 to about 100 alkylene oxide, particularly ethylene oxide residues; and non-alkoxylate emulsifiers including sugar mono-esters and polyglycerol mono-esters, hydrocarbyl, especially alkyl, polysaccharides; fatty acid glycerol esters where the fatty acid has 8 to 12 carbon atoms such as glycerol mono-laurate and fatty acid N-sugar amides such as glucamides. Relatively hydrophilic emulsifiers include alkoxylate emulsifiers with an average of from 2 to about 10 alkylene oxide, particularly ethylene oxide residues; glycerol esters where the fatty acid has 14 to 24 carbon atoms such as glycerol mono-stearate, -oleate, or -laurate; and anhydrosaccharide fatty esters such as sorbitan mono-stearate, -oleate, or -laurate.

The amount of emulsifier used is typically from about 0.02 to about 1.5%, more usually from about 0.025 to about 1.2%, particularly from about 0.025 to about 1%, by weight of the emulsion.

Where hydrophilic alkoxylate emulsifiers, especially those with HLB greater than about 12, are used, it is possible to obtain satisfactory emulsions with very low levels of emulsifier for example from as little as about 0.04 to about 0.1% by weight of the emulsion, and this forms a particular feature of the invention. Higher amounts of such emulsifiers can be used eg in the overall range about 0.04 to about 0.8%, particularly about 0.1 to about 0.6%, by weight. Where less hydrophilic alkoxylate emulsifiers are used as the primary emulsifier, the concentration used will typically be higher eg in the range from about 0.1 to about 1.5%, more usually from about 0.2 to about 1.2, particularly from about 0.5 to about 1%, by weight of the emulsion. Similarly where non-alkoxylate emulsifiers such as fatty acid esters, ethers, hemi-acetals or acetals of polyhydroxylic compounds, are used as the main emulsifier, the amount used will typically be from about 0.2 to about 1.2%, more usually from about 0.3 to about 1%, particularly from about 0.4 to 0.8%, by weight of the emulsion.

It is generally technically possible to freely combine non-ionic emulsifiers of the alkoxylate and non-alkoxylate types described above. Such combinations may be attractive where the emulsifier system includes a hydrophilic alkoxyate emulsifier, eg using a low HLB non-alkoxylate emulsifier in combination. However, hydrophilic non-alkoxylate emulsifiers, especially sugar mono-ester emulsifiers, are more expensive than typical alkoxylate emulsifiers and will usually be used only when it is desired to have an emulsifier stabiliser system which includes no derivatives of alkylene oxides.

The oil phase used will typically mainly be an emollient oil of the type widely used in personal care or cosmetic products. The emollient oil can and usually will be an oily material which is liquid at ambient temperature.

Suitable normally liquid emollient oils include non-polar oils, for example mineral or paraffin, especially isoparaffin, oils, such as that sold by Uniqema as Arlamol HD; or medium polarity oils, for example vegetable glyceride oils such as jojoba oil, animal glyceride oils, such as that sold by Uniqema as Estol 3609™ (triethylhexanoin), caprylic/capric triglycerides, synthetic oils, for example synthetic ester oils, such as isopropyl isostearate and propylene glycol isostearates sold by Uniqema as Prisorine 2021™ and Prisorine 2034™, respectively, C₁₂-C₁₅ alkyl benzoates or ether oils, particularly of two fatty e.g. C₈ to C₁₈ alkyl residues, such as that sold by Henkel as Eutanol G (octyl dodecanol), or silicone oils, such as dimethicione oil such as those sold by Dow Corning as DC2, cyclomethicone oil as sold by Dow Corning as DC245, or silicones having polyoxyalkylene side chains to improve their hydrophilicity or highly polar oils including alkoxylate emollients for example fatty alcohol propoxylates such as that sold by Uniqema as Arlamol E (stearyl alcohol 15-propoxylate) or alkyl carbonates such as Cetiol® CC (INCI: Dicaprylyl Carbonate) ex-Cognis. When non-polar oils are used it may be desirable to use relatively high concentrations of emulsifier, particularly high HLB emulsifier, in order to achieve suitably satisfactory emulsification, particularly to obtain small oil droplets.

Mixtures of emollient oils can and often will be used and in some cases solid emollient oils may dissolve wholly or partly in liquid emollient oils or in combination the freezing point of the mixture is suitably low. Where the emollient oil composition is a solid at ambient temperature, the resulting dispersion may technically not be an emulsion (although in most cases the precise phase of the oily disperse phase cannot readily be determined) but such dispersions behave as if they were true emulsions and the term emulsion is used herein to include such compositions.

The concentration of the oil phase may vary widely. Generally the oil phase concentration will be at least about 1%, and more usually at least about 3%, by weight of the final emulsion and in products as used the oil concentration can be as high as about 30%. Stable emulsions at oil phase content of upwards of 20% by weight have been obtained.

The composition of the emulsions of the invention, with regard to the main components, typically fall within the ranges in the tables below.

amount (wt %)

| Material | broad | preferred |
|---|---|---|
| oil | 1 to 80 | 1 to 10 |
| total emulsifier | 0.02 to 1.5 | 0.025 to 1.2 |
| high HLB emulsifier | 0.02 to 1.2 | 0.025 to 1.0 |
| *low HLB emulsifier | 0.01 to 1.2 | 0.03 to 0.15 |
| polysaccharide stabiliser | 0.02 to 0.5 | 0.01 to 0.25 |
| thickener (when used) | 0.1 to 10 | 0.25 to 7 |
| water** | to 100 | to 100 |

*used in combination with a high HLB emulsifier
**after allowing for minor components and additives.

The emulsions and formulations of this invention are typically near acid/base neutrality—their sensitivity to ionic materials is mentioned above. Moderate deviation from neutrality is possible without losing the stability advantages of the invention. Desirably the pH is from 4 to 9, more desirably 4.5 to 8 and particularly usefully from 6 to 8.

Many other components may be included in the emulsion compositions of the invention to make personal care or cosmetic formulations. These components can be oil soluble, water soluble or non-soluble. Among water soluble components, care may be needed with materials that provide electrolyte to the composition or cause marked shifts in pH (see above). Examples of such materials include:
preservatives and antimicrobials such as those based on parabens (alkyl esters of 4-hydroxybenzoic acid), phenoxyethanol, substituted ureas and hydantoin derivatives, eg those sold commercially under the trade names Germaben II Nipaguard BPX and Nipaguard DMDMH, isothiazolinones e.g. Kathon CG and Neolone 950 sold by Rohm and Haas, iodopropynyl butylcarbamate e.g. Glycacil 2000 sold by Lonza Inc., may be used usually in a concentration of from as low as about 5 ppm to 2% by weight of the emulsion depending upon the product and associated regulatory permitted levels of use;
perfumes, when used typically at a concentration of from 0.1 to 10% more usually up to about 5% and particularly up to about 2% by weight of the emulsion;
humectants or solvents such as alcohols, polyols such as glycerol and polyethylene glycols, when used typically at a concentration of from 1 to 10% by weight of the emulsion;
sunfilter or sunscreen materials including chemical sunscreens and physical sunscreens including those based on titanium dioxide or zinc oxide; when used typically at from 0.1% to 5% by weight of the emulsion (but noting that physical sunscreen materials are often dispersed using acrylic polyanionic polymers that may tend to destabilise the emulsions because they supply electrolyte);
alpha hydroxy acids such as glycolic, citric, lactic, malic, tartaric acids and their esters;
self-tanning agents such as dihydroxyacetone;
vitamins and their precursors including:
   a) Vitamin A eg as retinyl palmitate and other tretinoin precursor molecules,
   b) Vitamin B eg as panthenol and its derivatives,
   c) Vitamin C eg as ascorbic acid and its derivatives,
   d) Vitamin E eg as tocopheryl acetate,
   e) Vitamin F eg as polyunsaturated fatty acid esters such as gamma-linolenic acid esters;
botanical extracts such as Aloe Vera with beneficial skin care properties

The emulsions made in accordance with the invention can be used, as described above, as cosmetic or personal care products in themselves or can be fabricated into such products. For example, the emulsions of the invention may be used in spray applications, as body moisturisers, and in sun screen applications.

In particular, the emulsions can be used to impregnate wipe substrates as hereinbefore discussed. Such wipe substrates may be made from natural or synthetic materials both woven or non-woven, or combinations of such materials and/or combinations of both woven and non-woven layers. Natural materials include cellulose fibres and synthetic materials include fibres made from polyolefins, polyesters, rayon, polyamides, polyesteramides, polyvinyl alcohols or mixtures or two or more thereof. Preferred wipe substrates are selected from non-woven synthetic fibre based materials.

For application to wipe substrates, the emulsions made using the invention will typically contain a relatively low proportion of oil phase typically from 1 to 10%, preferably from 2 to 5%, more usually about 3% by weight of the emulsion. The amount of emulsion impregnated into wipe substrates will depend on the desired properties in the end product, but will typically be from 100 to 1000 g m⁻² of wipe substrate. The wipe substrates will typically have a base weight of from 30 to 100 g m⁻².

The wipes made using the invention may be used in cleansing applications such as personal hygiene and to remove make up or other cosmetics, for example.

Skin cleansing is a personal hygiene problem not always easily solved. Dry tissue products are the most commonly used cleansing products post-defecation or post-urine release. Dry tissue products are usually referred to as “toilet tissue” or “toilet paper”. As an alternative or accompaniment to the use of dry tissue, the use wet wipes for the purpose of cleaning the anus, the perinea, and the peri-anal body area after defecation is becoming increasingly popular to minimise common hygienic concerns. The use of such wipes is on behalf of babies and by children and adults.

Among those negatives associated with the failure of adequate cleansing are irritation, redness, desquamation, infections, unpleasant odor or other kinds of personal discomfort or health related issues.

People suffering from pathological conditions (such as hemorrhoids, fissures, cryptitis, etc.) are even more susceptible to those issues and discomfort. For them, as for any persons, cleansing must be efficient in terms of removal of fecal residues and gentle in terms of absence of irritation caused by the cleansing. Wet-wipes bring a response to that basic need.

In comparison to dry toilet paper, wet wipes have several benefits, including:
lubrication during the use of the wipe with a consequent reduction in the abrasiveness of the cleansing operation;
the softening and/or hydration of the residues, enhancing their removal from the skin;
the hydration of the skin tissue; and
the ability to deliver a soothing lotion to the skin that can remain on the skin after the cleansing operation.

Emulsions made using the invention are also particularly effective remove make up or other cosmetic preparations and can be broadly as efficient as the neat oil in removing oily make up e.g. mascara, particularly “waterproof mascara”. This is a surprising result as such emulsions in this use typically do not contain very high proportions of oil, typical amounts would be from 1 to 10, more usually from 2 to 5% by weight of the emulsion.

In wipe applications, the emulsions may be formulated with additional components such as fragrances, soothing agents, preservatives, rheology modifiers, moisturisers, texturers, colourants, medically active ingredients such as skin protection ingredients and healing promoters, and combinations thereof.

Preferred emollient oils used in wipe applications are silicone oils, synthetic ester oils and vegetable glyceride oils as described above.

Preferred emulsifiers, incorporated in the polysaccharide emulsion stabiliser used in the present invention, used in wipe applications are alkoxylates, particularly C₁₆-C₁₈ ethoxylates, particularly using a 2 mole ethoxylate as the low HLB component and a >20 mole ethoxylate (particularly a 100 mole ethoxylate) as the high HLB component.

Preferred soothing agents, ie compounds that have the ability to reduce the irritation or stinging/burning/itching effect of some chemicals, in addition to any soothing effect generated by the emollient oil selected, may include paraben-based preservative systems, antioxidants, buffer compounds and surfactants. Preferred soothing agents of the present invention are ethoxylated surface active compounds, more preferably those having an ethoxylation number below about 60; (b) polymers, more preferably polyvinylpirrolidone (PVP) and/or N-vinylcaprolactam homopolymer (PVC); and (c) phospholipids, more preferably phospholipids that are complexed with other functional ingredients as e.g., fatty acids, organosilicones. Most preferably the soothing agents of the present invention are selected from PEG40 hydrogenated castor oil, sorbitan isostearate, isoceteth-20, sorbeth-30, sorbitan monooleate, coceth-7, PPG-I-PEG-9 lauryl glycol ether, PEG-45 palm kernel glycerides, PEG-20 almond glycerides, PEG-7 hydrogenated castor oil, PEG-50 hydrogenated castor oil, PEG-30 castor oil, PEG-24 hydrogenated lanolin, PEG-20 hydrogenated lanolin, PEG-6 caprylic/capric glycerides, PPG-1 PEG-9 lauryl glycol ether, lauryl glucoside polyglyceryl-2 dipolyhydroxystearate, sodium glutamate, poly-vinylpyrrolidone, N-vinylcaprolactam homopolymer, sodium coco PG-dimonium chloride phosphate, linoleamidopropyl PG-dimonium chloride phosphate, sodium borageamidopropyl PG-dimonium chloride phosphate, N linoleamidopropyl PG-dimonium chloride phosphate dimethicone, cocamidopropyl PG-dimonium chloride phosphate, stearamidopropyl PG-dimonium chloride phosphate and stearamidopropyl PG-dimonium chloride phosphate (and) cetyl alcohol and combinations thereof.

Preferred preservatives are those sold under the trade names Phenonip ex-Clariant a combination of Nipaset and Phenoxetol ex-Clariant.

The present invention includes a wipe as herein described and an article of commerce comprising a container housing one or more wet wipes according to the invention.

The emulsifier and the polysaccharide emulsion stabiliser components used in the invention can be blended to provide a dry formulation that can be dispersed directly in polar emollient oil or in the non-aqueous liquid medium for subsequent combination with the emollient oil. Typically, these dry formulations include the solid components including the emulsifier and polysaccharide emulsion stabiliser. For such formulations, it is useful to use both high HLB and low HLB emulsifiers.

The composition of the dry formulation with regard to the main components, typically fall within the ranges in the table below.

(parts by wt)

| Material | broad | preferred |
|---|---|---|
| Xanthan | 2 to 10 | 3 to 8 |
| polyglucomannan | 2 to 10 | 3 to 8 |
| ratio | 1:4 to 4:1 | 1:2 to 2:1 |
| Xanthan:polyglucomannan | | |
| total emulslfier | 25 to 95 | 30 to 85 |
| high HLB emulsifier | 30 to 75 | 40 to 70 |
| * low HLB emulslfier | 5 to 40 | 10 to 30 |

* when used in combination

Preferably, the dry blended product is desirably a powder having a mean particle size of from about 100 to about 500 μm to facilitate dispersion of it in the emollient oil or non-aqueous liquid medium. To facilitate handling, eg to reduce the risk of powder combustion, the powder desirably contains little or no material having a much lower particle size. In particular, the proportion of particles of size lower than 50 μm is less than 10% (by weight), desirably less than 2%, particularly less than 1%.

The present invention will now be further described by way of illustration only with reference to the following Examples.

### EXAMPLES

The materials used in the Examples were as follows:
Emulsifier/polysaccharide emulsion stabiliser—a pre-formulated emulsifier and polysaccharide powder available as Arlatone V-150™ available from Uniqema. Arlatone V-150™ material contains Steareth-100, Steareth-2, Mannan, Xanthan Gum
Propylene glycol—available from BDH (solubility parameter 31)
Pricerine 9091™—glycerine available from Uniqema (solubility parameter 38).
Arlamol E™—stearyl alcohol 15-propoxylate oil available from Uniqema (solubility parameter 20.8).
Arlamol HD™T—isoparaffin oil available from Uniqema (solubility parameter 15.51).
Estol 3609™—triethylhexanoin oil available from Uniqema.
Prisorine 2021™—isopropyl isosterate oil available from Uniqema.
Prisorine 2034™—propylene glycol isosterate oil available from Uniqema (solubility parameter 19.54).
DC 245—a cyclopentasiloxane oil available from Dow Corning.
Nipaguard BPX™—phenoxyethenol, methylparaben, propylparaben, 2-bromo-2-nitropropane available from Clariant.
Nipasept™—methylparaben, ethylparaben, propylparaben available from Clariant.
Phenoxetol™—phenoxylethanol available from Clariant.

### Example 1

At ambient temperature, emulsions were made by adding the Pricerine 9091™ glycerine (1.0 wt %, 4.0 g) (Sample 1) or with the propylene glycol (1.0 wt %, 4.0 g) (Sample 2) to a 600 ml tall glass beaker and then adding the Arlatone V-150™ material (0.3 wt %, 1.2 g) to the beaker to disperse it in liquid phase by stirring at 300.rpm using a paddle stirrer having six blades (50 mm) diameter.

Emollient oil consisting of a mixture of Estol 3609™ oil (1.5 wt %, 6.0 g) Pricorine 2021™ oil (1.5 wt %, 6.0 g) (solubility parameter of the mixture was 19.04) was then added to the mixer.

Water (95.4 wt %, 381.6 g) was added slowly to the mixer during stirring until phase inversion occurred to form an oil-in-water emulsion, following which the remaining water was added at a faster rate. The Nipaguard BPX material (0.3 wt %, 1.2 g) was then added to the mixer.

The mixer was operated until the emulsion appeared to be homogenous (approximately 10 minutes).

This procedure was repeated but using stirring speeds of 500 and 700 rpm to generated further pairs of emulsions, Samples 3 (Pricerine 9091™ material) and 4 (propylene glycol) and Samples 5 (Pricerine 9091™ material) and 6 (propylene glycol).

The viscosities of the emulsions were measured after 24 hours using a Brookfield DV II+ viscometer using the conditions quoted in Table 1 below.

The stabilities of the emulsions was also visually inspected and they are quoted in Table 1 below.

In all instances, the Arlatone V-150™ material dispersed more quickly in the propylene glycol (Samples 2, 4 and 6) as compared to the Pricerine 9091™ glycerine (Samples 1, 3 and 5) and produced a more fluid mixture. Samples 2, 4 and 6 were also miscible with the oil phase, unlike Samples 1, 3 and 5. Visually, there was little difference in the appearances of the Samples.

TABLE 1

| | Viscosity | | Stability @ |
|---|---|---|---|
| | (24 hours) | Spindle, speed, | ambient |
| Sample | mPa · s | time & temperature | temperature |
| 1 | 1816 | Sp3, 6 rpm, 1 min@19.3° C. | >1 month |
| 3 | 1066 | Sp3, 6 rpm, 1 min@19.3° C. | >1 month |
| 5 | 1000 | Sp3, 6 rpm, 1 min@19.3° C. | >1 month |
| 2 | 2583 | Sp3, 6 rpm, 1 min@19.3° C. | >1 month |
| 4 | 1150 | Sp3, 6 rpm, 1 min@19.3° C. | >1 month |
| 6 | 2250 | Sp3, 6 rpm, 1 min@19.3° C. | >1 month |

### Example 2

Example 1 was repeated to produce Samples 7, 8 and 9 containing Arlatone V-150™ material (Sample 7—0.4 wt %, 1.6 g; Sample 8—0.5 wt %, 2.0 g; Sample 9—0.6 wt %, 2.4 g) in propylene glycol, the amount of water used being adjusted to accommodate the changes in the amounts of Arlatone V-150™ material used. A stirring rate of 500 rpm was used. The results are given in Table 2 below.

The Samples containing the increased levels of Arlatone V-150™ material were slightly whiter and less translucent.

TABLE 2

| | Viscosity | | Stability @ |
|---|---|---|---|
| | (24 hours) | Spindle, speed, | ambient |
| Sample | mPa · s | time & temperature | temperature |
| 7 | 2034 | Sp3, 6 rpm, 1 min@19.3° C. | >1 month |
| 8 | 2283 | Sp3, 6 rpm, 1 min@19.3° C. | >1 month |
| 9 | 2550 | Sp3, 6 rpm, 1 min@19.3° C. | >1 month |

### Example 3

Sample 2 of Example 1 was repeated (Sample 10). A comparative sample (Sample 11) was made using the same formulation and stirring rate (300 rpm), except that the Arlatone V-150™ material/propylene glycol were mixed in a separate vessel and the oil, water and Nipaguard BPX™ material were all added to the main mixer, following which the Arlatone V-150™ material/propylene glycol mixture was added to the main mixer. The results are given in Table 3.

TABLE 3

| | Viscosity | | |
|---|---|---|---|
| | (initial) | Spindle, speed, | |
| Sample | mPa · s | time & temperature | Stability |
| 10 | 648.3 | Sp3, 6 rpm, | >1 week - no visual |
| | | 1 min@18.5° C. | separation at |
| | | | temperatures of 50° C., |
| | | | 40° C., RT, 5° C., −5° C./ |
| | | | 40° C. cycling every 12 |
| | | | hours |
| 11 | 743.3 | Sp3, 6 rpm, | Creaming of large |
| | | 1 min@18.5° C. | droplets to the surface |
| | | | of the emulsion at 50° C. |
| | | | after 1 week, followed |
| | | | by creaming of the |
| | | | emulsion at 40° C., −5° C./ |
| | | | 40° C. cycling every 12 |
| | | | hours, after 3 weeks |

Sample 10 was similar to Sample 2 of Example 1. However, Sample 11 had oil droplets sufficiently large to be visible to the naked eye. Sample 10 had a white, translucent appearance as compared to Sample 11.

### Example 4

In a comparative experiment, the Arlatone V-150™ material (0.3 wt %, 1.2 g) was added directly to the oil phase (DC 245™ oil (3.0 wt %, 12.0 g)) together with Nipasept™ material (0.25 wt %, 1.0 g) and Phenoxetol™ material (0.8 wt %, 3.2 g) with stirring at 300 rpm. The water (95.65 wt %, 382.6 g) was added. Stirring was continued for about 10 minutes.

This experiment was repeated with the Arlatone V-150™ material (0.3 wt %, 1.2 g) being added directly to the oil phase (Arlamol HD™ ((3.0 wt %, 12.0 g) +) together with Nipasept™ material (0.25 wt %, 1.0 g) and Phenoxetol™ material (0.8 wt %, 3.2 g) with stirring at 300 rpm. The water (95.65 wt %, 382.6 g) was added. Stirring was continued for about 10 minutes.

In the first comparative experiment the Arlatone V-150 particles when added to DC 245 silicone oil wetted out and dispersed, as did the Nipasept™ material. However when the Phenoxetol™ material was added, it caused liquid globules to form containing clumped Arlatone V-150 particles. When the water was added the Arlatone V-150 particles began to swell; however clumps of un-wetted particles were still visible. After adding roughly one third of the water, clumps of un-wetted particles were still visible. After ten minutes stirring of the final emulsion, large clumps of un-wetted particles were still visible. Oil droplets were actually so large they were visible.

In the second comparative experiment, in which the oil phase used Arlamol HD oil, similar effects were noted during manufacture and resulted in an emulsion with slightly smaller but still visible oil droplets. After twenty four hours, the un-wetted particles had wetted out and formed clear gel clusters suspended within the emulsion.

### Example 5

Example 1 was repeated using a more polar emollient oil. Sample 12 was made using a stirring rate of 300 rpm and contained Arlatone V-150™ material (0.3 wt %, 1.2 g), propylene glycol (1.0 wt %, 4.0 g), Prisorine 2034 (3.0 wt %, 12.0 g), water (95.4 wt %, 381.69) and Nipaguard BPX™ material (0.3 wt %, 1.2 g).

The Sample 12 emulsion formed more readily than the emulsions formed using the Estol 3609™ and Prisorine 2021™ emollient oil mixture used in the earlier Examples. Sample 12 had a whiter appearance which may indicate the presence of smaller droplets. The results are given in Table 4.

TABLE 4

| | Viscosity | | | |
|---|---|---|---|---|
| | (initial) | Spindle, speed, | | Stability at ambient |
| Sample | mPa · s | time & temperature | pH | temperature |
| 12 | 640.0 | Sp1, 6 rpm, | 5.64 | >1 week |
| | | 1 min@20.0° C. | | |

### Example 6

Example 1 was repeated using a silicone oil (DC 245) (3.0 wt %, 24.0 g)—Sample 13 and Prisorine 2034 (3.0 wt %, 24.0 g)—Sample 14. The Samples were made using a stirring rate of 300 rpm and each contained Arlatone V-150™ material (0.3 wt %, 2.4 g), propylene glycol (1.0 wt %, 8.0 g), Nipasept™ material (0.225 wt %, 2.0 g) and phenoxyethanol (0.8 wt %, 6.4 g). The results are given in Table 5.

The Arlatone V-150 material and propylene glycol dispersion only partially dissolved, ie was less miscible, into the DC 245 oil—Sample 13—and was far less miscible compared to the Arlatone V-150 material and propylene glycol dispersion mixed into the Prisorine 2034 oil. A larger amount of water was needed to be added to Sample 13 before the emulsion appeared uniform. The Arlatone V-150 material swelled and formed clusters initially and two phases were visible. The addition of more water formed a uniform translucent emulsion with a viscosity build that appeared to be relatively stable and uniform. The formulation containing DC 245 oil appeared clear on the skin compared to the formulation containing Prisorine 2034 oil, which appeared white.

TABLE 5

| | Viscosity | Spindle, | | |
|---|---|---|---|---|
| | (initial) | speed, time & | | |
| Sample | mPa · s | temperature | pH | Stability |
| 13 | 878.3 | Sp3, 6 rpm, | 5.40 | >3 days - large visible |
| | | 1 min@20.0° C. | | droplets |
| 14 | 900.0 | Sp3, 6 rpm, | 5.47 | >3 days - stable at |
| | | 1 min@20.0° C. | | 50° C., 40° C., |
| | | | | RT, 5° C., −5° C./40° C. |
| | | | | cycling every 12 hours |

### Example 7

Example 4 was repeated using Arlamol E oil (3.0 wt % 24.0 g)—Sample 15 and Prisorine 2034 oil (3.0 wt %, 12.0 g)—Sample 16. The Samples were made using a stirring rate of 300 rpm, the Arlatone V-150™ material (0.3 wt %, 12 g) being added to the oil, which contained Nipasept™ material (0.25 wt %, 1.0 g) and phenoxyethanol (0.8 wt %, 3.2 g). The balance of water added was 95.65 wt %, 382.6 g. The results are given in Table 6.

For Sample 15, the majority of the Arlatone V-150 powder dissolved directly into the Arlamol E oil after 10 minutes stirring at 300 rpm, the oil phase blend was slightly cloudy with some clumps of Arlatone V-150 particles still visible. As the water was added the initial viscosity increased (still fluid) until the inversion which produced a more fluid translucent emulsion.

For Sample 16, the majority of the Arlatone V-150 powder dissolved directly into the Prisorine 2034 oil after ten minutes stirring, this time producing a clear oil blend with the Arlatone V-150 particles still remaining being better dispersed than in Sample 15. As the water was added, again an initial viscosity increase was visible until the inversion and then a white emulsion was formed.

For both Samples, any remaining Arlatone V-150 particles that have not dissolved into the oil phase swelled and were incorporated once the water phase has been added. During the experiment, the oil phase was continually stirred not allowing any un-dissolved Arlatone V-150 particles to sediment.

TABLE 6

| | Viscosity (initial) | Spindle, speed, |
|---|---|---|
| Sample | mPa · s | time & temperature |
| 15 | 630.3 | Sp3, 6 rpm, 1 min@20.0° C. |
| 16 | 640.0 | Sp3, 6 rpm, 1 min@20.0° C. |

1. A method of making an oil-in-water emulsion comprising:
dispersing an oil emulsifier and a polysaccharide emulsion stabiliser, which stabiliser comprises a Xanthan polysaccharide and a polyglucomannan polysaccharide, in an oil, to form an oil-based dispersion; and
combining the oil-based dispersion with water, preferably at low temperature and under low shear conditions, to form an oil-in-water emulsion.

2. A method according to claim 1 in which the oil emulsifier and the polysaccharide emulsion stabiliser components are dispersed directly into a relatively polar oil to form the oil-based dispersion.

3. A method according to claim 2 in which the oil has a Hansen and Beerbower solubility parameter of at least 19 but less than 45.

4. A method according to claim 1 in which the polysaccharide emulsion stabiliser and optionally the oil emulsifier are dispersed in a relatively polar nonaqueous liquid medium, which medium then being mixed with the oil prior to the step of combining the oil-based dispersion with the water to form the emulsion.

5. A method according to claim 4 in which the oil has a Hansen and Beerbower solubility parameter of less than 19 and the non-aqueous liquid medium has a Hansen and Beerbower solubility parameter of at least 19 but less than 45.

6. A method according to claim 5 in which the oil is selected from silicone oils.

7. A method according to claim 4 in which the oil has a Hansen and Beerbower solubility parameter of at least 19 but less than 45 and the non-aqueous liquid medium has a Hansen and Beerbower solubility parameter of at least 19 but less than 45.

8. A method of making an oil-in-water emulsion comprises the steps of:
(a) dispersing a polysaccharide emulsion stabiliser and optionally an oil emulsifier, wherein said polysaccharide emulsion stabiliser comprises a Xanthan polysaccharide and a polyglucomannan polysaccharide, either in:
(i) an emollient oil with a Hansen and Beerbower solubility parameter of at least 19 but less than 45; or
(ii) a non-aqueous liquid medium with a Hansen and Beerbower solubility parameter of at least 19 but less than 45 and combining the dispersion in said medium with an oil that has a Hansen and Beerbower solubility parameter of at least 19 but less than 45; or
(iii) a non-aqueous liquid medium with a Hansen and Beerbower solubility parameter of at least 19 but less than 45 and combining the dispersion in said medium with an oil that has a Hansen and Beerbower solubility parameter of less than 19;
(b) combining water with the oil-based dispersion formed in step (a), preferably at low temperature and under low shear conditions, to form an oil-in-water-emulsion, and
(c) continuing to mix the emulsion formed in step (b), preferably under low shear conditions, until homogenous.

9. A method according to claim 8 in which the oil in step (a)(iii) is selected from silicone oils.

10. A method of making an oil-in-water emulsion comprises the steps of:
(a) dispersing a polysaccharide emulsion stabiliser and optionally an oil emulsifier, wherein said polysaccharide emulsion stabiliser comprises a Xanthan polysaccharide and a polyglucomannan polysaccharide, in a non-aqueous liquid medium with a Hansen and Beerbower solubility parameter of at least 19 but less than 45 and combining the dispersion in said medium with an oil selected from the group consisting of silicone oils;
(b) combining water with the oil-based dispersion formed in step (a), preferably at low temperature and under low shear conditions, to form an oil-in-water-emulsion
(c) continuing to mix the emulsion formed in step (b), preferably under low shear conditions, until homogenous.

11. A method of making a wet wipe, comprising the steps of:
making an oil-in-water emulsion according to the method claimed in claim 1; and
applying the oil-in-water emulsion to a wipe substrate.

12. An emollient oil composition comprising emollient oil in which is substantially dispersed oil emulsifier and a polysaccharide emulsion stabiliser wherein said polysaccharide emulsion stabiliser comprises a Xanthan polysaccharide and a polyglucomannan polysaccharide.

13. An emollient oil composition comprising emollient oil and a non-aqueous liquid medium with a Hansen and Beerbower solubility parameter of at least 19 but less than 45 in which is dispersed oil emulsifier and a polysaccharide emulsion stabiliser wherein said polysaccharide emulsion stabiliser comprises a Xanthan polysaccharide and a polyglucomannan polysaccharide.

14. A composition according to claim 12 in which the emollient oil has a Hansen and Beerbower solubility parameter of less than 19.

15. A composition according to claim 14 in which the oil is selected from silicone oils.

16. A composition according to claim 12 in which the emollient oil has a Hansen and Beerbower solubility parameter of at least 19 but less than 45.

17. A composition according to claim 12 which contains not greater than 1% by weight of water.

18. A composition according to claim 17 which is essentially water free.

19. A composition according to claim 12 which contains at least 70% by weight of oil and, when present, non-aqueous liquid medium, more preferably at least 80% by weight of oil and, when present, non-aqueous liquid medium, the balance being water and, optionally, other additives.

20. A wipe comprising a wipe substrate impregnated with an oil-in-water emulsion made in accordance with claim 1.

21. A wipe comprising a wipe substrate impregnated with an oil-in-water emulsion, said emulsion comprising from 1 to 10%, by weight of the emulsion of an oil phase and further comprising from about 0.1 to 0.5% by weight of the emulsion of oil emulsifier and a polysaccharide emulsion stabiliser components, wherein said polysaccharide emulsion stabiliser comprises a Xanthan polysaccharide and a polyglucomannan polysaccharide.

22. An article of commerce comprising a container and, contained therein, a wipe according to claim 20.
